SEQUENCE LISTING (31 sequences)

SEQ ID NO: 1 - protein, 124 aa, Artificial Sequence
  HER3-MF3178
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDHGSRHFWSYWGFDYWGQGTLVTVSS

SEQ ID NO: 2 - protein, 124 aa, Artificial Sequence
  HER3-MF3163
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAKDSYSRHFYSWWAFDYWGQGTLVTVSS

SEQ ID NO: 3 - protein, 124 aa, Artificial Sequence
  HER3-MF6061
QVQLVQSGAEVKKPGASVKVSCKASGYTFTGYYMHWVRQAPGQGLEWMGWINPQSGGTNYAQKFKGRVTMTRDTSTSTAYMELSRLRSDDTAVYYCARDHGSRHFWSYWGFDYWGQGTLVTVSS

SEQ ID NO: 4 - protein, 120 aa, Artificial Sequence
  PD1-MF6930
EVQLVQSGAEVKKPGSSMKVSCKASGGTFSNYVISWVRQAPGQGLEWMGMIIPVFETATYEKKFQGRVTIIADKSTSTVYMELSSLRSEDAAVYYCARGTVEATLLFDYWGQGTLVTVSS

SEQ ID NO: 5 - protein, 120 aa, Artificial Sequence
  PD1-MF6256
EVQLVQSGAEVKKPGSSMKVSCKASGGTFSSYVISWVRQAPGQGLEWMGMIIPVFDTSSYEKKFQGRITIIADKSTSTVYLELSSLRSEDAAVYYCARGTVEATLLFDFWGQGTLVTVSS

SEQ ID NO: 6 - protein, 122 aa, Artificial Sequence
  HER2-MF1849
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKGDYGSYSSYAFDYWGQGTLVTVSS

SEQ ID NO: 7 - protein, 121 aa, Artificial Sequence
  LGR5-MF7423
QVQLVQSGAEVKKPGSSVKVSCKASGGTFSNYAISWVRQAPGQGLEWMGSIIPILGTTDHAQKFQDRVTITAGKSSNTAYMDLSSLRSDDTAVYYCARERIAARLDYFDSWGQGTLVTVSS

SEQ ID NO: 8 - protein, 120 aa, Artificial Sequence
  EGFR-MF3755
QVQLVQSGSELKKPGASVKISCKASGYDFTNYAMNWVRQAPGHGLEWMGWINANTGDPTYAQGFTGRFVFSLDTSVSTAYLQISSLKAEDSAVYYCTRERFLEWLHFDYWGQGTLVTVSS

SEQ ID NO: 9 - protein, 121 aa, Artificial Sequence
  LGR5-MF7428
QVQLVQSGAEVKKPGSSVKVSCKASGGTFSNYAISWVRQAPGQGLEWMGSIIPILGTTDHAQKFQDRVTITADKSSKTTYMELNSLRSDDTAVYYCARELIAARLDYFDSWGQGTLVTVSS

SEQ ID NO: 10 - protein, 121 aa, Artificial Sequence
  LGR5-MF7533
QVQLVQSGAEVKKPGSSVKVSCKASGGTFSNYAISWVRQAPGQGLEWMGSIIPILGTTDHAQKFQDRVTITAGKSSNTAYMDLSSLRSDDTAVYYCARERIAARLDYFDSWGRGTLVTVSS

SEQ ID NO: 11 - protein, 127 aa, Artificial Sequence
  LGR5-MF7532
EVQLVQSGSKLKKPGASVKVSCKASGYTFTSYTMNWVRQAPGQGLEWMGWINTDTGDPTYAQGFTGRFVFSLDTSVSTAFLQINSLKAEDTAVYYCARGDCDSTSCYRYSYGYEDYWGRGTLVTVSS

SEQ ID NO: 12 - protein, 125 aa, Artificial Sequence
  EGFR-MF4280
QVQLVQSGAEVKKPGASVKVSCKVSGYTLTELSMHWVRQAPGKGLEWMGGFDPEYGKTFFAQNFQGRVTMTEDTSADTAYMELSSLRSEDTAVYYCATEGYYETTTYYYNLFDSWGQGTLVTVSS

SEQ ID NO: 13 - protein, 121 aa, Artificial Sequence
  HER2-MF3958
QVQLVQSGAEVKKPGASVKLSCKASGYTFTAYYINWVRQAPGQGLEWIGRIYPGSGYTSYAQKFQGRATLTADESTSTAYMELSSLRSEDTAVYFCARPPVYYDSAWFAYWGQGTLVTVSS

SEQ ID NO: 14 - protein, 119 aa, Artificial Sequence
  EGFR-MF4003
QVQLVQSGSELKKPGASVKVSCKASGYTFPSFAMNWLRQAPGQGLEWMGWITTNTGDPTYAQGFSGRFVFSLDTSVSTAYLQISSLKAEDTAVYYCARVYNWIRGFDYWGQGTLVTVSS

SEQ ID NO: 15 - protein, 10 aa, Artificial Sequence
  Synthetic sequence
AAAAAAAAAA

SEQ ID NO: 16 - protein, 10 aa, Artificial Sequence
  Synthetic sequence
AAAAAAAAAA

SEQ ID NO: 17 - protein, 121 aa, Artificial Sequence
  HER2-MF3025
QVQLKQSGAELVRPGTSVKLSCKASGYTFTGYYINWVKQRPGQGLEWIARIYPGSGYTYYNEKFKGKATLTAEESSNTAYMHLSSLTSEDSAVYFCARPHYGYDDWYFAVWGTGTTVTVSS

SEQ ID NO: 18 - protein, 120 aa, Artificial Sequence
  EGFR-MF4010
QVQLVQSGSELKKPGASVKVSCKASGYTFTNNAMNWVRQAPGQGLEWMGWINTITGDPSYAQGFTGRFVFSLDTSVNTAYLQISSLKAEDTAVYYCAREEFLEWLFFDYWGQGTLVTVSS

SEQ ID NO: 19 - protein, 121 aa, Artificial Sequence
  TIM3-MF6501
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDSNAWDSMAFDYWGQGTLVTVSS

SEQ ID NO: 20 - protein, 122 aa, Artificial Sequence
  HER2-MF1898
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDGFRRTTLSGFDYWGQGTLVTVSS

SEQ ID NO: 21 - protein, 122 aa, Artificial Sequence
  HER2-MF2032
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDYYRRTARAGFDYWGQGTLVTVSS

SEQ ID NO: 22 - protein, 6 aa, Artificial Sequence
  VL CDR1
QSISSY

SEQ ID NO: 23 - protein, 7 aa, Artificial Sequence
  VL CDR2
AASSLQS

SEQ ID NO: 24 - protein, 7 aa, Artificial Sequence
  VL CDR3
QQSYSTP

SEQ ID NO: 25 - protein, 95 aa, Artificial Sequence
  IgVk1-39*01
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 26 - protein, 107 aa, Artificial Sequence
  Common Light Chain IgKV1*39/jk1
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 27 - protein, 108 aa, Artificial Sequence
  Common Light Chain IgKV1*39/jk5
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 28 - protein, 109 aa, Artificial Sequence
  IgG1 CH1
ASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCCVECPPC

SEQ ID NO: 29 - protein, 111 aa, Artificial Sequence
  IgG1 CH2
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 30 - protein, 107 aa, Artificial Sequence
  IgG1 CH3 KK
GQPREPQVYTKPPSREEMTKNQVSLKCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 107 aa, Artificial Sequence
  IgG1 CH3 DE
GQPREPQVYTDPPSREEMTKNQVSLTCEVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2022/0127375 A1
(43) Pub. Date: Apr. 28, 2022

(54) PRODUCING COMPOSITIONS COMPRISING TWO OR MORE ANTIBODIES

(71) Applicant: Merus N.V., Utrecht (NL)

(72) Inventors: Robert Paul DOORNBOS, Utrecht (NL); Alexander Berthold Hendrik BAKKER, Utrecht (NL)

(73) Assignee: Merus N.V., Utrecht (NL)

(21) Appl. No.: 17/431,041

(22) PCT Filed: Feb. 13, 2020
(86) PCT No.: PCT/NL2020/050080

§ 371 Date: Aug. 13, 2021

(30) Foreign Application Priority Data
Feb. 14, 2019 (EP) 19157286.6
Jun. 5, 2019 (EP) 19178542.7

(51) Int. Cl. C07K 16/32; C07K 1/20; C07K 1/18; C07K 16/28; C07K 1/22

(52) U.S. Cl. CPC C07K 16/32; C07K 1/20; C07K 1/18; C07K 2317/526; C07K 1/22; C07K 2317/31; C07K 16/2863

(57) ABSTRACT

The invention relates to means and methods of producing at least two antibodies. Methods may include providing cells with nucleic acid that encodes the antibodies; culturing said cells; collecting the antibodies from the culture; and separating produced antibodies from half antibodies by ion exchange chromatography (IEX). In some embodiments the antibodies exhibit IEX retention times that that deviate by 10% or less from the average of the retention times of the individual antibodies under the IEX conditions used. The invention also relates to compositions of antibodies thus produced. In some aspects the invention relates to compositions comprising 2-10 recombinant antibodies characterized in that the IEX retention times of at least two of said antibodies deviate by 10% or less from the average of the retention times of the individual antibodies under the IEX conditions. It also relates to compositions comprising 2-10 recombinant antibodies characterized in that the pI of at least two of said antibodies differ by 0.4 units or less from the average pI of said at least two antibodies.

The invention relates to the field of antibodies, in particular to the field of therapeutic antibodies. The antibodies can be used in the treatment of humans. More in particular the invention relates to the production and/or purification of multiple antibodies. A single host cell can produce the multiple antibodies. The antibodies can also be produced by a mixture of host cells that each produces one of the antibodies. The invention also relates to methods for producing compositions comprising such antibodies and for purifying such antibodies.

Polyclonal antibodies are typically collected from the blood of a subject. An advantage of polyclonal antibodies is that pathogens are attacked via multiple targets and epitopes. An advantage of monoclonal or recombinant antibodies is the well characterized specificity and function allowing such antibodies to be employed as precision medicaments with a well-defined action and toxicity spectrum.

The specificity of a monoclonal antibody can also be a drawback, particularly when multiple targets need to be addressed. It is possible to reduce this disadvantage by adding more antibodies to the medicament, but considering that even a single therapeutic antibody can be expensive it is envisioned that the costs for such multiclonal cocktails can quickly become prohibitive.

The development of bi-, tri and other multispecific antibodies has been successful in introducing some aspects of a polyclonal antibody to antibody therapeutics. Apart from the increase in the number of targets it has also successfully introduced additional functionality not previously attainable with classical monospecific mono- or polyclonal antibodies. The use of multiple bi-, tri and other multispecific antibodies within the same treatment may provide yet further benefits.

The present invention provides an advance in the art by describing a robust and economic method for the purification of multiple antibody therapeutics produced from a single host cell or alternatively, a mixture of host cells. The invention is particularly useful for the economic production of collections of two or more antibodies, preferably multispecific antibodies.

### SUMMARY OF THE INVENTION

The invention provides a method of producing at least two multispecific antibodies comprising
providing cells with nucleic acid that enables the multispecific antibodies;
culturing said cells;
collecting the multispecific antibodies from the culture; and
separating produced multispecific antibodies from half-antibodies, and optionally monospecific antibodies and/or other undesired antibody product related impurities by ion exchange chromatography (IEX);
   the method characterized in that the multispecific antibodies exhibit IEX retention times that deviate by 10% or less from the average of the retention times of the individual multispecific antibodies under the IEX conditions used. The retention times of the respective half antibodies and optionally monospecific antibodies and/or other undesired antibody product related impurities are preferably outside the range spanned by the retention times of the multispecific antibodies.

The invention also provides a method of producing at least two multispecific antibodies comprising
providing a cell with nucleic acid that encodes the multispecific antibodies;
culturing said cell;
collecting the multispecific antibodies from the culture; and
separating produced multispecific antibodies from half-antibodies, and optionally monospecific antibodies and/or other undesired antibody product related impurities by ion exchange chromatography (IEX);
   the method characterized in that the multispecific antibodies exhibit IEX retention times that are essentially the same under the IEX conditions used. The retention times of the respective half antibodies and optionally monospecific antibodies and/or other undesired antibody product related impurities are preferably outside the range spanned by the retention times of the antibodies.

The invention further provides a method of producing at least two antibodies, wherein the antibodies comprise a monospecific and/or a multispecific antibody comprising
providing cells with nucleic acid that encodes the antibodies;
culturing said cells;
collecting the antibodies from the culture; and
separating produced antibodies from half-antibodies by ion exchange chromatography IEX):
   the method characterized in that the antibodies exhibit IEX retention times that are essentially the same under the IEX conditions used. The retention times of the respective half antibodies and optionally monospecific antibodies are preferably outside the range spanned by the retention times of the antibodies.

The invention also provides a composition comprising 2-10 recombinant antibodies obtainable by a method as described herein.

Also provided is a composition comprising 2-10 recombinant antibodies such as multispecific antibodies characterized in that the IEX retention times of at least two of said antibodies deviate by 10% or less from the average of the retention times of the individual antibodies under the IEX conditions used.

Further provided is a composition comprising 2-10 recombinant antibodies characterized in that the IEX retention times of at least two of said antibodies are essentially the same.

Also provided is a composition comprising 2-10 recombinant antibodies characterized in that the isoelectric point (pI) of at least two of said antibodies preferably differ by 0.4 units, 0.3, 0.2 and preferably 0.1 units or less from the average pI of said at least two antibodies. The pI of each of said at least two antibodies preferably differs by 0.25 units or less from each other.

### DETAILED DESCRIPTION OF THE INVENTION

The term “antibody” as used herein means a proteinaceous molecule belonging to the immunoglobulin class of proteins, containing one or more domains that bind an epitope on an antigen, where such domains are or derived from or share sequence homology with the variable region of an antibody. Antibodies are typically made of basic structural units—each with two heavy chains and two light chains. Antibodies for therapeutic use are preferably as close to natural antibodies of the subject to be treated as possible (for instance human antibodies for human subjects). Antibodies with extended heavy and/or light chain variable region are also included herein. An antibody according to the present invention is not limited to any particular format or method of producing it.

Half antibodies are heavy and light chain combinations that are not associated with another heavy and light chain combination and that do not form an interface with another variable region or variable region-like polypeptide. Other undesired antibody product related impurities can be free light chains that are not associated with a heavy chain, free heavy chains that are not associated with a light chain or another heavy chain, or incompletely assembled antibodies lacking a light chain.

Suitable cells for antibody production are known in the art and include a hybridoma cell, a Chinese hamster ovary (CHO) cell, an NS0 cell or a PER-C6 cell, or a variety of other cell lines known to persons of ordinary skill in the art. Various institutions and companies have developed cell lines for the large scale production of antibodies, for instance for clinical use. Non-limiting examples of such cell lines are CHO cells, NS0 cells or PER.C6 cells or HEK293 cells, among others. In a particularly preferred embodiment said cell is a human cell. Preferably a cell that is transformed by an adenovirus E1 region or a functional equivalent thereof. In a preferred embodiment said cell is a CHO cell or a variant thereof. Preferably a variant that makes use of a Glutamine synthetase (GS) vector system for expression of an antibody. In one preferred embodiment, the cell is a CHO cell.

The cells can be provided with the nucleic acid that encodes the antibodies. The cells will express, assemble and excrete the formed antibodies into the supernatant of the cell cultures. Introduction of a single heavy and light chain (or rather the nucleic acid coding for it), leads to the production of a monoclonal antibody that has two heavy chains that are each associated with a light chain.

A method of the invention can be performed with mixtures of cells comprising two or more kind of cells that each produces a different antibody. An advantage of using such a mixture is that downstream processing of collected antibodies can be streamlined more efficiently. A further advantage of a method is that antibody products are collected and purified as one. Also, a mixture of two or more antibodies produced by a method of the invention could reduce the number of tests required for obtaining regulatory approval when compared to the number of tests required for each of the antibodies separately.

In a further embodiment the cells are a homogenous collection of cells consisting essentially of replicas of a single cell provided with nucleic acid that encodes the respective antibodies. Co-expression of several heavy chains in a cell allows for various combinations of heavy chains. The combination with additional light chains increases the number of combinations. Various methods have been developed to favor the formation of specific combinations over others. Heavy chain variants have been produced that specifically facilitate the formation of heavy chain heterodimers over homodimers or vice versa heavy chain homodimers over heterodimers. Heavy chains with specific homo- or heterodimerization domains reduce the number antibodies that are being produced by such cells and/or increase the level of the preferred antibody over the alternative combination (e.g., higher production of a heterodimer over a homodimer).

A method of the invention is particularly suited for the production of two or more multispecific antibodies, including bispecific antibodies. Various methods for the production of bispecific antibodies exist in the art. One approach utilizes a common light chain that can form functional variable domains with different heavy chains. One preferred method for producing bispecific antibodies includes use of a transgenic animal harboring a common chain in its genome, such that immunization of such animal with an antigen results in a repertoire of antibodies that are specific to said antigen based on the non-common chain, wherein the repertoire consists of a variety of antibodies comprising said common chain and a rearranged cognate chain. One animal can be immunized with different antigens, or different animals can be immunized with each of the respective antigen separately. Nucleic acids encoding the non-common chain or the variable region thereof may be obtained from the animal(s), for example the B-cells, spleen or lymph tissue. These can be used to generate nucleic acid that can express the respective non-common chains which can then be introduced into the production cell. The common chain can be introduced at the same or a different time. The nucleic acid can be incorporated into the nucleus, and preferably into the genome of the host cell, such that the host cell produces multispecific antibodies or multimers targeting the multiple antigens, for which the transgenic animal(s) had been immunized (see for instance, WO2009/157771 which is incorporated by reference herein for the purpose of the generation of variable regions that can combined with a common chain to produce functional variable domains that are specific for different targets and/or different epitopes).

A cell that produces a common light chain and two different heavy chains that each can form a functional variable domain with the common light chain, produces among others a bispecific antibody with two different heavy and light chain combinations. Similarly, a cell that produces a common light chain and three or more different heavy chains can form multispecific antibodies capable of targeting three or more antigens, or a combination of two or more multispecific antibodies capable of targeting three or more antigens. Presently it is possible to build on the standard format of antibodies (i.e. a constant part and two variable domains) and add further binding domains. As such multispecific antibodies can be made that have one or more single chain Fv with additional binding specificities attached to the constant or one or more of the variable domains of an antibody. It is also possible to produce heavy chains with two or more variable regions. The additional heavy chain regions can advantageously associate with different or common light chain variable regions. Reference is made to U.S. 62/650,467 which is incorporated by reference herein.

When the cell produces two or more multispecific antibodies it can, in cases, also produce some amount of half-antibodies and antibodies with identical heavy chains or homodimers. The amount of the latter can be reduced by including heterodimer formation promoting modifications in the heavy chains. As indicated above, various methods for inducing heterodimerization of heavy chains exist. The respective domains with the modifications are collectively referred to as heterodimerization domains. Heavy chains that have heterodimerization domains that favor interaction are said to have compatible heterodimerization domains. The compatible heterodimerization domains are preferably compatible immunoglobulin heavy chain CH3 heterodimerization domains. The art describes various ways in which such CH3 heterodimerization of heavy chains can be achieved.

One preferred method for producing bispecific antibodies is disclosed in U.S. Pat. Nos. 9,248,181 and 9,358,286. Specifically, preferred changes to produce essentially only bispecific full length IgG molecules are the amino acid substitutions L351K and T366K (EU numbering) in the first CH3 domain (the ‘KK-variant’ heavy chain) and the amino acid substitutions L351D and L368E in the second domain (the ‘DE-variant’ heavy chain), or vice versa. As previously described, the DE-variant and KK-variant preferentially pair to form heterodimers (so-called‘DEKK’ bispecific molecules). Homodimerization of DE-variant heavy chains (DEDE homodimers) or KK-variant heavy chains (KKKK homodimers) hardly occur, including due to strong repulsion between the charged residues in the CH3-CH3 interface between identical heavy chains.

In the present invention it is preferred that the cells are provided with nucleic acid that encodes a common light chain. Various methods are available to the skilled person to produce antibodies that have different heavy chain variable regions but the same light chain variable region. WO2004/106375 describes phage libraries using a common light chain variable region. Phage selections yield variable domains with the same light chain variable region but different heavy chain variable regions. Further, non-human animals having a common chain and different cognate chains are described in WO2009/157771. Antibody selections in such animals yield variable domains with the same or similar common chain variable regions but different cognate chain variable regions. WO2004/106375 and WO2009/157771 are incorporated by reference herein. The publications are referred to specifically for the generation of antibodies and nucleic acid encoding such antibodies that have the same or similar common chain variable regions and different cognate chain variable regions, preferably a common light chain variable region and different heavy chain variable regions.

In one preferred embodiment the cell produces two or more heavy chains and a common light chain. The respective heavy and light chains may have one or more variable regions associated with the respective chains. In a preferred embodiment the cell produces three or more heavy chains. One of the three heavy chains preferably contains one part of a compatible heterodimerisation domain. The two or more other heavy chains preferably comprise the other part of the compatible heterodimerization domain. If the first heavy chain is symbolically represented by the letter “A”, and the two other heavy chains by the letters “B” and “C”, the particular combination of heterodimerization domains leads to the formation of predominantly the combinations AB and AC. The combinations AA, BB, CC, and BC are disfavored by the inclusion of the heterodimerization domains. Such a cell is effective producing only the two bispecific antibodies AB and AC (see FIG. 1). In the present invention it is preferred that the cell produces the two antibodies by producing at least 3 heavy chains. In a preferred embodiment one of said heavy chains contains one part of a compatible heterodimerization domain and said two or more other heavy chains preferably comprise the other part of the compatible heterodimerization domain. A heavy chain that is shared by two or more bi- or multispecific antibodies or multimers in a composition preferably has the CH3 DE part of the heterodimerization domain. The other heavy chains in the bi- or multispecific antibodies preferably have the CH3 KK part.

The at least two antibodies are preferably multispecific antibodies, preferably bispecific antibodies. In a preferred embodiment at least two of said antibodies share an identical heavy chain. The cell can produce several series of bispecific antibodies by including different heterodimerization domains in the heavy chains. Such implementations can lead to the predominant production of bispecific antibodies with heavy chain combinations AB and CD. The combination AB could be favored by a DE/KK heterodimerization domain as referred to herein above, and the CD by the incorporation of a “knob in hole” heterodimerization domain, or other heterodimerization features known in the art, for example through charge engineering. A shared heavy chain between different bi- or multispecific multimer or antibody combinations with the shared heavy chain can be made by providing a shared heavy chain with one part of a heterodimerization domain and the various combination chains with the complementary part of the heterodimerization domain. For instance the CH3 DE part in the shared heavy chain and the CH3 KK part in the combination chains. One shared heavy chain and two combination heavy chains would, in this setting, result in the production of bi- or multispecific multimer or antibodies with heavy chain combinations AB and AC by the cell (or AxBC and AxDE for multispecific multimers). Other possible combinations are AB. AE. CD and CF; or AB. AE. AG and CD etc.

Antibodies generally have a distinctive isoelectric point (typically in the range of pH 6-10) compared to other host cell proteins. The antibodies, such as multispecific antibodies, as well as multispecific multimers, can be purified with a relatively high purity through methods disclosed herein. Methods may comprise a number of steps such as culturing the host cell, undergoing harvest clarification, followed by protein capture. IEX chromatography such as anion exchange chromatography may be used to remove host cell DNA, cation exchange chromatography (CIEX) can be used, for instance to remove host cell protein, leached protein A and potential aggregates. Additional steps may be included such as virus filtration or hydrophobic interaction chromatography.

Antibodies are typically excreted by the producing cells. Harvesting of such antibodies typically involves collection of the cell supernatant followed by several purification steps to remove cell debris or aggregates of which the presence is not desired. Harvest clarification can involve filtration, centrifugation or a combination thereof of the culture supernatant of antibody producing cells. Antibody protein capture is typically done by affinity purification. This can be done in several ways. Often this involves the purification using columns that have recombinant protein A, protein G or protein L, which are bacterial proteins with a known high specific binding capacity for antibodies. Presently various optimized mutants are available that more specifically bind antibodies. For instance a recombinant protein A is available of which non-essential domains have been removed, a recombinant protein G is available with its albumin binding site deleted and also modified protein L is available. Bound antibodies can be collected by elution for one or more of such columns. Anion- and cation-exchange chromatography can be used to further purify the preparations, for instance, purifying bispecific or multispecific antibodies from half-antibodies and/or homodimer antibodies and/or other undesired antibody product related impurities, if any. Hydrophobic interaction chromatography (HIC) is commonly used as an alternative polishing step in antibody purification processes. HIC offers an orthogonal selectivity to ion exchange chromatography and can be an effective step for aggregate clearance and host cell protein reduction. In the present invention, HIC may be used for analytical purposes, after purification of the two or more bi- or multispecific antibodies or multimers is accomplished, to then quantify the two or more species having similar, preferable essentially the same retention time in the IEX and/or similar, preferably essentially the same pI. Thus, HIC is used to quantify the relative quantities of the purified molecules.

A hydrophobic interaction resin is selected as the stationary phase and the pH and/or conductivity of the mobile phase is modulated to achieve the required selectivity. Antibodies typically attract positive charge at lower pH, which has an effect on its polarity and overall surface hydrophobicity. pH conditions can be selected that allow the segregation of the two or more antibodies in the preparation.

In some embodiments collected antibodies are first separated from other proteins by affinity purification, preferably by protein A extraction. Subsequently the affinity purified antibodies are run on an anion exchange column under conditions that collect the antibodies in the flow-through fraction. Antibodies can subsequently run on one or more CIEX columns.

A preferred method for producing at least two antibodies is done by producing them by cells as a single composition that comprises the two or more antibodies.

A method of producing at least two antibodies preferably comprises culturing the host cell producing the two or more antibodies, collection of the supernatant of the cells, treating the supernatant harvest in a harvest clarification process, preferably comprising filtration with a pore size cut-off that traps aggregates such as cells or cell debris and allows passage of the antibody products. The antibody is collected from the cell culture fluid by affinity chromatography using protein A. The antibody bound to the protein resin is eluted from the chromatography column following exposure to low pH and the eluate is subsequently neutralized using a suitable buffer.

As used herein, the term “isoelectric point (pI)” means the pH at which the average net charge of the protein surface, that is, the potential of the electric double layer of the protein, is 0. In other words, the term means the point at which a group of proteins is dissociated so that the numbers of cations and anions are equal, and thus the net charge of the protein is 0.

As used herein, “pI” is calculated based on the primary amino acid according to ExPASy. ProtParam tool, using default parameters as of the earliest filing date (priority date) of the present application. The ProtParam is a tool which allows the computation of various physical and chemical parameters for a given protein stored in Swiss-Prot or TrEMBL or for a user entered protein sequence. The computed parameters include the theoretical pI. Casteiger E., Hoogland C., Gattiker A., Duvaud S., Wilkins M. R., Appel R. D., Bairoch A.; Protein Identification and Analysis Tools on the ExPASy Server; (In) John M. Walker (ed): The Proteomics Protocols Handbook, Humana Press (2005) pp. 571-607.

A protein's net surface charge changes with pH in a manner that is dictated by a protein's pI. At a pH equal to a protein's pI, the protein will carry no net charge. At a pH below the pI, the protein will carry a net positive charge. If the buffer pH is raised above a protein's pI, it will carry a net negative charge.

A protein's pI may be determined by its primary amino acid sequence and can thus be calculated, a buffer can then be chosen that ensures a known net charge for a protein of interest. A negatively charged cation exchange resin is thus chosen when the protein of interest carries a net positive charge at the working pH.

Proteins with different pI values will have varying degrees of charge at a given pH and thereby have different affinities for the positively charged surface groups on the particles of the anion exchange media; therefore, different proteins will bind to the resin with different strengths, facilitating their separation. Thus, by generating heterodimeric polypeptides having distinctive pI relative to the homodimer and half-antibody contaminants and/or other antibody product related impurities, the heterodimeric polypeptides can be readily purified using standard elution techniques, for example, by applying pI gradient, or by applying a salt or conductivity gradient at a fixed pH, or a combination of a pH and a conductivity gradient. In embodiments of the invention the constant parts and the light chains in the antibodies essentially have the same sequence in the different antibodies. Such antibodies typically differ essentially only in the amino acid sequence of the heavy chain variable regions. For such antibodies it is typically sufficient to calculate the pI of the heavy chain variable regions as indicated herein. The calculations and criteria are then set by using the pI of the heavy chain variable regions instead of the pI of the (half)-antibody. The average pI of the heavy chain variable regions of the antibody is indicative for the retention time of the antibody in a CIEX-column. The antibodies can, if any, have the same or different heterodimerization domains. They preferably have the same heterodimerization domains. The antibodies may further differ somewhat in the exact amino acid sequence in the constant parts.

For the ion-exchange chromatography (IEX) step(s), this is a process that separates ions and polar molecules based on their affinity to the ion exchanger. It works on a variety of charged molecules-including large proteins, small nucleotides, and amino acids. IEX is often used in protein purification. The water-soluble and charged proteins form ionic bonds with the insoluble stationary phase. The bound molecules then can be eluted and collected using an eluent with higher concentration of ions and/or a different pH. The concentration of salt or the pH can be changed in a stepwise fashion; by gradually changing the mobile phase of the chromatography run, or a combination thereof. The two types of ion chromatography are anion-exchange and cation-exchange. Cation-exchange chromatography or (CIEX) is preferred in the present invention. Antibody CIEX is preferably performed at a physiological pH. The pH is typically with a range of 5-9, preferably 6-8.

CIEX is typically used when the molecule of interest is positively charged under the pH used for chromatography. The molecule is positively charged because the pH for chromatography is less than the pI of the molecule. In this type of chromatography, the stationary phase is negatively charged and positively charged molecules are loaded to be attracted to it. Anion-exchange chromatography is when the stationary phase is positively charged and negatively charged molecules (meaning that pH for chromatography is greater than the pI) are loaded to be attracted to it.

An antibody such as a multispecific antibody is typically bound to the IEX column in a binding stage under conditions that promote the binding of the antibody such as a multispecific antibody to the substrate. IEX columns are typically subsequently washed to remove unbound material. Elution from the column is done in an elution stage. The retention time of an antibody such as a multispecific antibody is typically calculated from the start of the elution stage. It is the amount of time that the antibody spends on the column when the elution phase has started. If a sample contains several compounds, each compound in the sample will typically spend a different amount of time on the column according to its chemical composition i.e. each will have a different retention time. Retention times are usually quoted in units of seconds or minutes.

The retention times of the antibodies such as multispecific antibodies are preferably essentially the same in a method of the invention. Different antibodies can have different retention times on the same column and conditions. In the present invention it has been found that antibodies such as multispecific antibodies may be selected or designed to have IEX retention times that are sufficiently close to allow the co-purification of two or more antibodies such as two or more multispecific antibodies in a single IEX chromatography run. Retention times that deviate by 10% or less from the average of the retention times of the individual antibodies are typically sufficiently close to allow co-purification of two or more antibodies such as two or more multispecific antibodies in a single IEX chromatography run.

A suitable CIEX HPLC method for antibody purification and/or analytics according to the invention uses TSKgel SP-STAT (7 μm particle size, 4.6 mM I.D.×10 cm L, Tosoh 21964) series of ion exchange columns. The columns are packed with non-porous resin particles for speed and high resolution analysis, as well as isolation, of biomolecules. The particles in TSKgel STAT columns contain an open access network of multi-layered ion-exchange groups for loading capacity, while the particle size makes these columns suitable for HPLC and FPLC systems.

A suitable method involves the equilibration of the TSKgel SP-STAT (7 μm particle size, 4.6 mM I.D×10 cm L. Tosoh 21964) using Buffer A (Sodium Phosphate buffer, 25 mM. pH 6.0), after which antibodies are displaced from the column by increasing salt concentration and running a gradient of Buffer B (25 mM Sodium Phosphate, 1 mM NaCl, pH 6.0). Flow rate is set at 0.5 mL/min. The injection sample mass for the test samples and controls is 10 μg and injection volumes 10-100 μl. The chromatograms are analyzed for peak patterns, retention times and peak areas for the major peaks observed based on the 220 nm results. The method can be scaled for larger amounts of antibody.

Typical graphs of CIEX chromatography runs of antibody preparations are depicted in FIG. 2. The antibodies for this run were collected from transfected cells as indicated in the examples and purified from many other proteins in the medium by with a protein A column. Antibodies were eluted by acid elution followed by neutralization and buffer exchange to PBS pH 7.4 as indicated in the examples. A sample of the antibody preparation was subsequently loaded onto the CIEX column. After washing associated proteins were eluted by applying a salt gradient. The CIEX conditions were the same for the sample in FIG. 2A and FIG. 2B. The retention time is calculated for the top of the peak of the bispecific antibody. The retention time of two or more antibodies such as multispecific antibodies, preferably deviate by 10% or less from the average of the retention times of the two or more antibodies. A deviation of more than 10% typically results in inefficient separation of the antibodies from half antibodies and optionally in the case of multispecific antibodies from homodimers and/or other antibody product related impurities. In a preferred embodiment the retention times of two or more antibodies deviate by 9% or less from the average of the retention times of the two antibodies. Preferably by 8%, 7%, 6%, or 5% or less from the average of the retention times of the two or more antibodies. In a preferred embodiment the retention times of two or more antibodies deviate by 4% or less from the average of the retention times of the two or more antibodies. Preferably 3% or less, Preferably 2% or less. Increasingly similar retention times typically allows for increasingly more efficient separation of the multispecific antibodies from half antibodies and optionally homodimers and/or other antibody product related impurities, and thus allow more clean collection of the two antibodies in fractions of the IEX column.

In means and methods of the invention the average retention times of the two or more antibodies such as the two or more multispecific antibodies is calculated for the antibodies that are to be co-purified or collected. Thus in embodiments wherein the antibodies to be purified are multispecific antibodies, the average retention times of the two or more antibodies is calculated based on the multispecific antibodies. The retention times of antibodies that are not to be collected such as homodimer antibodies are not used for the calculation of the average.

Antibodies such as multispecific antibodies can be selected for co-purification in a method of the invention by selecting antibodies such as multispecific antibodies that have essentially the same IEX retention times under the conditions used for the IEX. The antibodies such as multispecific antibodies can also be tailored through appropriate modification of one or more variable regions to have essentially the same IEX retention times under the same or similar conditions used for the IEX.

In one embodiment the produced antibodies sought to be co-purified such as bi- and/or multispecific antibodies, have similar pls. The isoelectric point (pI) of at least two of said antibodies preferably differ by 0.4 units, 0.3, 0.2 and preferably 0.1 units or less from the average pI of said at least two antibodies. The pI of each of said at least two antibodies preferably differs by 0.25 units or less from each other.

A small to no difference in the pIs of the antibodies typically allows a good co-purification. Advantageously the pIs of the respective half-antibodies in an antibody differ more from the average. This difference facilitates a good separation of the half-antibodies from the “co”-migrating full antibodies in the CIEX chromatography step.

In embodiments wherein the antibodies sought to be co-purified are bi- or multispecific antibodies it is preferred that the pIs of the variable domains within each of the antibodies sought to be co-purified differ by more than 0.2, preferably 0.3, preferably 0.4, 0.5, 0.6, 0.7, 1.0, 1.2, 1.4 preferably more than 1.8 or 2.0 units from the average of the pIs of the variable domains of the other antibody(ies) to be co-purified. In this embodiment the difference in the pIs of the variable domains in an antibody is preferably at least 0.2 units greater than the difference between “x” and “y”, preferably it is at least 0.3, 0.4, 0.5, preferably at least 0.6, 0.7, 0.8, 0.9. 1.0, 1.2, 1.5, 2.0 or 2.5 greater than the difference between “x” and “y”, where “x” is the average of the pIs of the two variable domains of a first of said antibodies and “y” is the average of the pIs of the two variable domains of a second of said antibodies A difference as mentioned in the pIs of the variable domains within an antibody is typically indicative for a good separation of the antibody product related impurities from the antibodies sought to be co-purified and/or of a good separation from mono-specific antibodies.

Some compositions comprising two or more bi- or multispecific antibodies have constant regions and light chains that are similar in amino acid sequence, or have an essentially identical amino acid sequence. Such two or more bi- and/or multispecific antibodies typically differ from each other essentially only in the amino acid sequence of the variable domains, or essentially only in heavy chain variable regions. In such cases it is often not necessary to determine the pI of the entire antibody. Instead the pI of the variable domains and/or the pIs of the heavy chain variable regions can be determined. This provides a means of evaluating whether the antibodies may migrate close together in a CIEX chromatography step, in other words whether the antibodies have retention times that are sufficiently the same to allow co-purification.

In one embodiment of a method or composition as disclosed herein two or more bi- or multispecific antibodies have constant regions and light chains that have the same amino acid sequence, or have essentially identical amino acid sequences. The two or more bi- or multispecific antibodies can be co-purified in a CIEX chromatography step when the average pIs of the variable domains in each antibody differ by 0.7 units or less from the average pI of the variable domains of the respective antibodies sought to be co-purified. In a preferred embodiment the average “x” of the pIs of the two variable domains of a first of said antibodies and the average “y” of the pIs of the two variable domains of a second of said antibodies differ by 0.6 units or less, preferably 0.5 units or less different from the average of “x” and “y” of the first and second antibody sought to be co-purified. “x” and “y” are preferably 0.4; preferably 0.3; preferably 0.2 and preferably 0.1 units or less different from the average of “x” and “y” of the first and second antibody sought to be co-purified. Such bi- and/or multispecific antibodies, typically have retention times that are essentially the same. In this embodiment the constant regions of the antibodies are essentially the same. The pIs, and in particular the averages “x” and “y” of such antibodies are indicative for the pIs of the respective antibodies as a whole. In this embodiment it is preferred that the pIs of the variable domains within each of the antibodies sought to be co-purified differ by more than 0.2. preferably 0.3, preferably 0.4, 0.5, 0.6, 0.7, 1.0, 1.2, 1.4 preferably more than 1.8 or 2.0 units from the average of the pIs of the variable domains in the antibody. In this embodiment the difference in the pIs of the variable domains in an antibody is preferably at least 0.2 units greater than the difference between “x” and “y”, preferably it is at least 0.3, 0.4, 0.5, preferably at least 0.6, 0.7, 0.8, 0.9, 1.0, 1.2, 1.5, 2.0 or 2.5 greater than the difference between “x” and “y”. A difference as mentioned in the pIs of the variable domains within an antibody is typically indicative for a good separation of half-antibodies from the antibodies sought to be co-purified and/or of a good separation from mono-specific antibodies.

In one embodiment of a method or composition as disclosed herein two or more bi- or multispecific antibodies have constant regions and light chains that have the same amino acid sequence, or have essentially identical amino acid sequences. The two or more bi- or multispecific antibodies can be co-purified in a CIEX chromatography step when the average pIs of the heavy chain variable regions in each antibody differ by 0.7 units or less from the average pI of the heavy chain variable regions of the respective antibodies sought to be co-purified. In a preferred embodiment the average “m” of the pIs of the two heavy chain variable regions of a first of said antibodies and the average “n” of the pIs of the two heavy chain variable regions of a second of said antibodies differ by 0.6 units or less, preferably 0.5 units or less different from the average of “m” and “n” of the first and second antibody sought to be co-purified. “m” and “n” are preferably 0.4; preferably 0.3; preferably 0.2 and preferably 0.1 units or less different from the average of “m” and “n” of the first and second antibody sought to be co-purified. Such bi- and/or multispecific antibodies, typically have retention times that are essentially the same. In this embodiment the constant regions of the antibodies are essentially the same. The pIs, and in particular the averages “m” and “n” of such antibodies are indicative for the pIs of the respective antibodies as a whole. In this embodiment it is preferred that the pIs of the heavy chain variable regions within each of the antibodies sought to be co-purified differ by more than 0.2, preferably 0.3, preferably 0.4, 0.5, 0.6, 0.7, 1.0, 1.2, 1.4 preferably more than 1.8 or 2.0 units from the average of the pIs of the heavy chain variable regions in the antibody. In this embodiment the difference in the pIs of the heavy chain variable regions in an antibody is preferably at least 0.2 units greater than the difference between “m” and “n”, preferably it is at least 0.3, 0.4, 0.5, preferably at least 0.6, 0.7, 0.8, 0.9, 1.0, 1.2, 1.5, 2.0 or 2.5 greater than the difference between “m” and “n”. A difference as mentioned in the pIs of the heavy chain variable regions within an antibody is typically indicative for a good separation of half-antibodies from the antibodies sought to be co-purified and/or of a good separation from mono-specific antibodies.

The antibodies such as multispecific antibodies, can have, or be selected to have, heavy and light chain combinations (half antibodies) or homodimers (e.g., monospecific antibodies) or other antibody product related impurities that have retention times that are significantly different from the retention times of the full antibodies or desired antibodies such as multispecific antibodies, under the IEX conditions used. In embodiments wherein the cell expresses a common light chain, the selection is typically on the heavy chain. The heavy chains can be modified such that the half antibodies or homodimers have very different retention times. In a preferred embodiment the retention times of the half antibodies and/or homodimers differ by more than 10% of the average of the retention times of the respective antibodies or multispecific antibodies. In a preferred embodiment the average pI of individual heavy and light chain combinations of an antibody sought not to be co-purified differs by more than 0.5 units from the average pI of said heavy and light chains of the least two antibodies that are to be co-purified.

The invention further provides a composition comprising 2-10 recombinant antibodies obtainable by a method as described herein. Also provided is a composition comprising 2-10 recombinant antibodies characterized in that the IEX retention times of at least two of said antibodies are essentially the same.

The invention further provides a composition comprising 2-10 recombinant antibodies characterized in that the pI of at least two of said antibodies differ by 0.4 units, 0.3, 0.2 and preferably 0.1 units or less from the average pI of said at least two antibodies. The pI of each of said at least two antibodies preferably differs by 0.25 units or less from each other.

The invention further provides a composition comprising 2-10 recombinant antibodies characterized in that the average “x” of the pIs of the two variable domains of the first antibody and the average “y” of the pIs of the two variable domains of the second antibody are 0.7, 0.6 and preferably 0.5 units or less different from the average of “x” and “y” of the first and second antibody sought to be co-purified. “x” and “y” are preferably 0.4; preferably 0.3; preferably 0.2 and preferably 0.1 units or less different from the average of “x” and “y” of the first and second antibody sought to be co-purified. Such antibodies such as multispecific antibodies, typically have retention times that are essentially the same. In this embodiment the constant regions of the antibodies are essentially the same. The pIs, and in particular the average of the pIs of the different variable domains of the antibody each comprising a heavy chain variable region and a light chain variable region is indicative for the pI of the antibody as a whole.

The invention further provides a composition comprising 2-10 recombinant antibodies characterized in that the average “m” of the pIs of the two heavy chain variable regions of the two variable domains of the first antibody and the average “n” of the pIs of the two heavy chain variable regions of the two variable domains of the second antibody are 0.7, 0.6 and preferably 0.5 units or less different from the average of “m” and “n” of the first and second antibody sought to be co-purified. “m” and “n” are preferably 0.4; preferably 0.3; preferably 0.2 and preferably 0.1 units or less different from the average of “m” and “n” of the first and second antibody sought to be co-purified. Such antibodies such as multispecific antibodies, typically have retention times that are essentially the same. In this embodiment the constant regions and the light chain variable regions of the antibodies are essentially the same. The pIs, and in particular the average of the pIs of the different heavy chain variable regions of the antibody is indicative for the pI of the antibody as a whole.

In a preferred embodiment the IEX retention times and/or the pI are preferably essentially the same for all of the antibodies to be collected in the composition. In a preferred embodiment at least two of the antibodies are bispecific antibodies. Preferably at least two of said antibodies share an identical heavy chain.

In some embodiments, the common light chain variable region of one or both VH/VL binding regions comprises a germline IgVκ1-39*01 variable region V-segment. In certain embodiment, the light chain variable region of one or both VH/VL binding regions comprises the kappa light chain V-segment IgVκ1-39*01. IgVκ1-39 is short for Immunoglobulin Variable Kappa 1-39 Gene. The gene is also known as Immunoglobulin Kappa Variable 1-39; IGKV139; IGKV1-39. External Ids for the gene are HGNC: 5740; Entrez Gene: 28930; Ensembl: ENSG00000242371. The amino acid sequence for the V-region is provided in SEQ ID NO: 25. The V-region can be combined with one of five J-regions. Preferred J-regions are jk1 and jk5, and the joined sequences are indicated as IGKV1-39/jk1 and IGKV1-39/jk5; alternative names are IgVκ1-39*01/IGJκ1*01 or IgVκ1-39*01/IGJκ5*01 (nomenclature according to the IMGT database worldwide web at imgt.org). In certain embodiments, the light chain variable region of one or both VH/VL binding regions comprises the kappa light chain IgVκ1-39*01/IGJκ1*01 or IgVκ1-39*01/IGJκ1*05 (SEQ ID NO: 26 and SEQ ID NO: 27 respectively).

In some embodiments, the light chain variable region of one or both VH/VL binding regions of a bispecific antibody comprises an LCDR1 comprising the amino acid sequence QSISSY (SEQ ID NO: 22), an LCDR2 comprising the amino acid sequence AAS, and an LCDR3 comprising the amino acid sequence QQSYSTP (SEQ ID NO: 24) (i.e., the CDRs of IGKV1-39 according to IMGT). In some embodiments, the light chain variable region of one or both VH/VL binding regions of a bispecific antibody comprises an LCDR1 comprising the amino acid sequence QSISSY (SEQ ID NO: 22), an LCDR2 comprising the amino acid sequence AASLQS (SEQ ID NO: 23), and an LCDR3 comprising the amino acid sequence QQSYSTP (SEQ ID NO: 24).

In some embodiments, one or both VH/VL binding regions of a bispecific antibody comprise a light chain variable region comprising an amino acid sequence that is at least 90%, preferably at least 95%, more preferably at least 97%, more preferably at least 98%, more preferably at least 99% identical or 100% identical to the amino acid sequence of set forth in SEQ ID NO: 26. In some embodiments, one or both VH/VL binding regions of a bispecific antibody comprise a light chain variable region comprising an amino acid sequence that is at least 90%, preferably at least 95%, more preferably at least 97%, more preferably at least 98%, more preferably at least 99% identical or 100% identical to the amino acid sequence of set forth in SEQ ID NO: 27.

For example, in some embodiments, the variable light chain of one or both VH/VL binding regions of a bispecific antibody can have from 0 to 10, preferably from 0 to 5 amino acid insertions, deletions, substitutions, additions or a combination thereof with respect to SEQ ID NO: 26 or SEQ ID NO: 27. In some embodiments, the light chain variable region of one or both VH/VL binding regions of a bispecific antibody comprises from 0 to 9, from 0 to 8, from 0 to 7, from 0 to 6, from 0 to 5, from 0 to 4, preferably from 0 to 3, preferably from 0 to 2, preferably from 0 to 1 and preferably 0 amino acid insertions, deletions, substitutions, additions with respect to the indicated amino acid sequence, or a combination thereof.

In other embodiments, the light chain variable region of one or both VH/VL binding regions of a bispecific antibody comprises the amino acid sequence of SEQ ID NO: 26 or SEQ ID NO: 27. In certain embodiments, both VII/VL binding regions of a bispecific antibody comprise identical VL regions. In one embodiment, the VL of both VH/VL binding regions of a bispecific antibody comprises the amino acid sequence set forth in SEQ ID NO: 26. In one embodiment, the VL of both VH/VI, binding regions of a bispecific antibody comprises the amino acid sequence set forth in SEQ ID NO: 27.

Bispecific antibodies such as those disclosed in the methods herein can be provided in a number of formats. Many different formats of bispecific antibodies are known in the art. For example, bispecific antibody formats that are not classical antibodies with two VH/VL combinations have at least a variable domain comprising a heavy chain variable region and a light chain variable region. This variable domain may be linked to a single chain Fv-fragment, monobody, a VH and a Fab-fragment that provides the second binding activity.

Bispecific antibodies such as those disclosed in methods provided herein are generally of the human IgG subclass (e.g., for instance IgG1, IgG2, IgG3, IgG4). In certain embodiments, the antibodies are of the human IgG1 subclass. Full length IgG antibodies are preferred because of their favorable half-life and for reasons of low immunogenicity. Accordingly, in certain embodiments, the bispecific antibodies are full length IgG molecules. In an embodiment, the bispecific antibodies are full length IgG1 molecules.

In certain embodiments, antibodies comprises a fragment crystallizable (Fc). The Fe of bispecific antibodies are preferably comprised of a human constant region. A constant region or Fc of bispecific antibodies may contain one or more, preferably not more than 10, preferably not more than 5 amino-acid differences with a constant region of a naturally occurring human antibody. For example, in certain embodiments, each Fab-arm of the bispecific antibodies may further include an Fe-region comprising modifications promoting the formation of the bispecific antibody, modifications affecting Fc-mediated effector functions, and/or other features described herein.

In one aspect, provided is a pharmaceutical composition comprising two or more antibodies as defined herein and a pharmaceutically acceptable carrier. As used herein, the term “pharmaceutically acceptable” means approved by a government regulatory agency or listed in the U.S. Pharmacopeia or another generally recognized pharmacopeia for use in animals, particularly in humans, and includes any and all solvents, salts, dispersion media, coatings, antibacterial and antifungal agents, isotonic and absorption delaying agents, and the like that are physiologically compatible. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil, glycerol polyethylene glycol ricinoleate, and the like. Water or aqueous solution saline and aqueous dextrose and glycerol solutions may be employed as carriers, particularly for injectable solutions. Liquid compositions for parenteral administration can be formulated for administration by injection or continuous infusion. Routes of administration by injection or infusion include intravesical, intratumoral, intravenous, intraperitoneal, intramuscular, intrathecal and subcutaneous. Depending on the route of administration (e.g., intravenously, subcutaneously, intra articularly and the like) the active compound may be coated in a material to protect the compound from the action of acids and other natural conditions that may inactivate the compound.

Pharmaceutical compositions suitable for administration to human patients are typically formulated for parenteral administration. e.g., in a liquid carrier, or suitable for reconstitution into liquid solution or suspension for intravenous administration. The compositions may be formulated in dosage unit form for ease of administration and uniformity of dosage.

Also included are solid preparations which are intended for conversion, shortly before use, to liquid preparations for either oral or parenteral administration. Such liquid forms include solutions, suspensions and emulsions.

A “bispecific antibody” is an antibody as described herein wherein one domain of the antibody binds to a first antigen whereas a second domain of the antibody binds to a second antigen, wherein said first and second antigens are not identical. The term “bispecific antibody” also encompasses antibodies wherein one heavy chain variable region/light chain variable region (VH/VL) combination binds a first epitope on an antigen and a second VH/VL combination that binds a second epitope. The term further includes antibodies wherein VI is capable of specifically recognizing a first antigen and the VL, paired with the VH in an immunoglobulin variable region, is capable of specifically recognizing a second antigen. The resulting VH/VL pair will bind either antigen 1 or antigen 2. Such so called “two-in-one antibodies”, described in for instance WO 2008/027236, WO 2010/108127 and Schaefer et al (Cancer Cell 20, 472-486, October 2011). A bispecific antibody according to the present invention is not limited to any particular bispecific format or method of producing it.

“Percent (%) identity” as referring to nucleic acid or amino acid sequences herein is defined as the percentage of residues in a candidate sequence that are identical with the residues in a selected sequence, after aligning the sequences for optimal comparison purposes. The percent sequence identity comparing nucleic acid sequences is determined using the AlignX application of the Vector NTI Program Advance 10.5.2 software using the default settings, which employ a modified ClustalW algorithm (Thompson, J. D., Higgins. D. G., and Gibson T. J. (1994) Nuc. Acid Res. 22: 4673-4680), the swgapdnarnt score matrix, a gap opening penalty of 15 and a gap extension penalty of 6.66. Amino acid sequences are aligned with the AlignX application of the Vector NTI Program Advance 11.5.2 software using default settings, which employ a modified ClustalW algorithm (Thompson, J. D., Higgins, D. G., and Gibson T. J., 1994), the blosum62mt2 score matrix, a gap opening penalty of 10 and a gap extension penalty of 0.1.

The term ‘common light chain’ as used herein refers to the two light chains (or the VI, part thereof) in the bispecific antibody. The two light chains (or the VL part thereof) may be identical or have some amino acid sequence differences while the binding specificity of the full length antibody is not affected. The terms ‘common light chain’, ‘common VL’, ‘single light chain’, ‘single VL’, with or without the addition of the term ‘rearranged’ are all used herein interchangeably. “Common” also refers to functional equivalents of the light chain of which the amino acid sequence is not identical. Many variants of said light chain exist wherein mutations (deletions, substitutions, insertions and/or additions) are present that do not influence the formation of functional binding regions. The light chain of the present invention can also be a light chain as specified herein above, having from 0 to 10, preferably from 0 to 5 amino acid insertions, deletions, substitutions, additions or a combination thereof. It is for instance within the scope of the definition of common light chains as used herein, to prepare or find light chains that are not identical but still functionally equivalent, e.g., by introducing and testing conservative amino acid changes, changes of amino acids in regions that do not or only partly contribute to binding specificity when paired with the heavy chain, and the like. The term ‘full length IgG’ or ‘full length antibody’ according to the invention is defined as comprising an essentially complete IgG, which however does not necessarily have all functions of an intact IgG. For the avoidance of doubt, a full length IgG contains two heavy and two light chains. Each chain contains constant (C) and variable (V) regions, which can be broken down into domains designated CH1, CH2, CH3, VH, and CL, VL. An IgG antibody binds to antigen via the variable region domains contained in the Fab portion, and after binding can interact with molecules and cells of the immune system through the constant domains, mostly through the Fe portion. Full length antibodies according to the invention encompass IgG molecules wherein mutations may be present that provide desired characteristics. Full length IgG should not have deletions of substantial portions of any of the regions. However, IgG molecules wherein one or several amino acid residues are deleted, without essentially altering the binding characteristics of the resulting IgG molecule, are embraced within the term “full length IgG”. For instance, such IgG molecules can have a deletion of between 1 and 10 amino acid residues, preferably in non-CDR regions, wherein the deleted amino acids are not essential for the binding specificity of the IgG.

As an antibody typically recognizes an epitope of an antigen, and such an epitope may be present in other compounds as well, antibodies according to the present invention that “specifically recognize” an antigen may recognize other compounds as well, if such other compounds contain the same kind of epitope. Hence, the terms “specifically recognizes” with respect to an antigen and antibody interaction does not exclude binding of the antibodies to other compounds that contain the same kind of epitope.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein (so-called linear and conformational epitopes). Epitopes formed from contiguous, linear amino acids are typically retained on exposure to denaturing solvents, whereas epitopes formed by tertiary folding, conformation are typically lost on treatment with denaturing solvents. An epitope may typically include 3, 4, 5, 6, 7, 8, 9, 10, 11, 12, 13, 14 or 15 amino acids in a unique spatial conformation. Methods of determining spatial conformation of epitopes are known to persons of ordinary skill in the art and include techniques in the art for example, x-ray crystallography. HDX-MS and 2-dimensional nuclear magnetic resonance, pepscan, and alanine scan depending on the nature of the epitope (see, e.g., Epitope Mapping Protocols in Methods in Molecular Biology. Vol. 66. G. E. Morris. Ed. (1996)).

For the purpose of clarity and a concise description features are described herein as part of the same or separate embodiments, however, it will be appreciated that the scope of the invention may include embodiments having combinations of all or some of the features described.

In order that the present description may be more readily understood, certain terms are first defined. Additional definitions are set forth throughout the detailed description. Unless stated otherwise, all technical and scientific terms used herein have the same meaning as commonly understood by one of ordinary skill in the art, and conventional methods of immunology, protein chemistry, biochemistry, recombinant DNA techniques and pharmacology are employed.

As used herein, the singular forms “a”, “an” and “the” include plural referents unless the context clearly dictates otherwise. Use of the term “including” as well as other forms, such as “include”. “includes”, and “included”, is not limiting.

### BRIEF DESCRIPTION OF THE DRAWINGS

FIG. 1

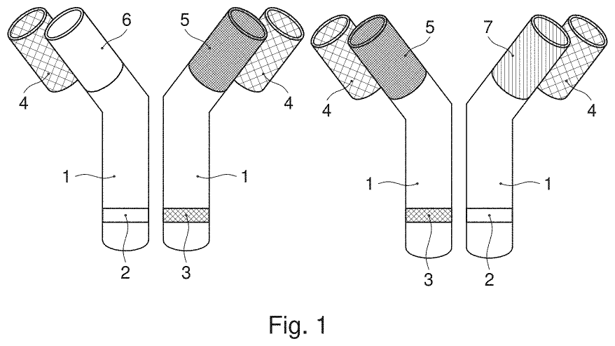

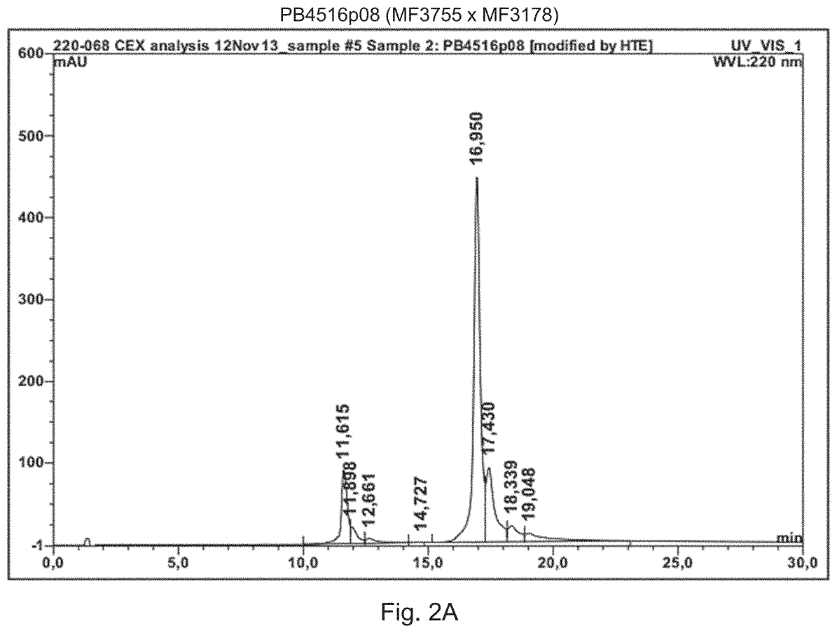

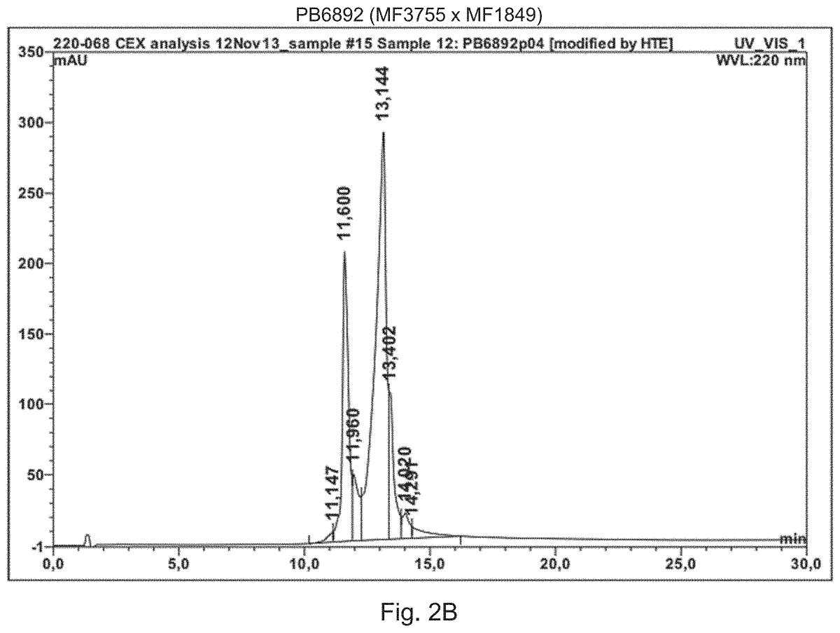

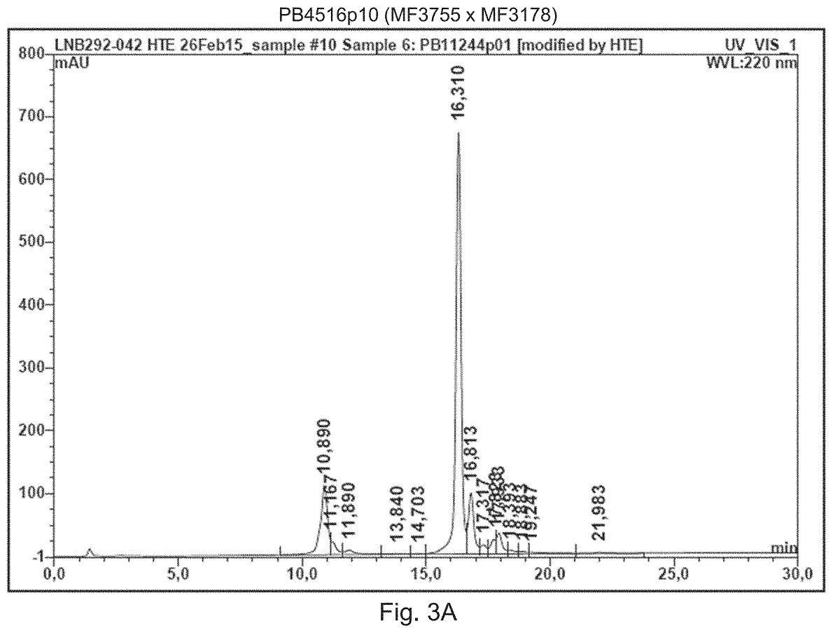

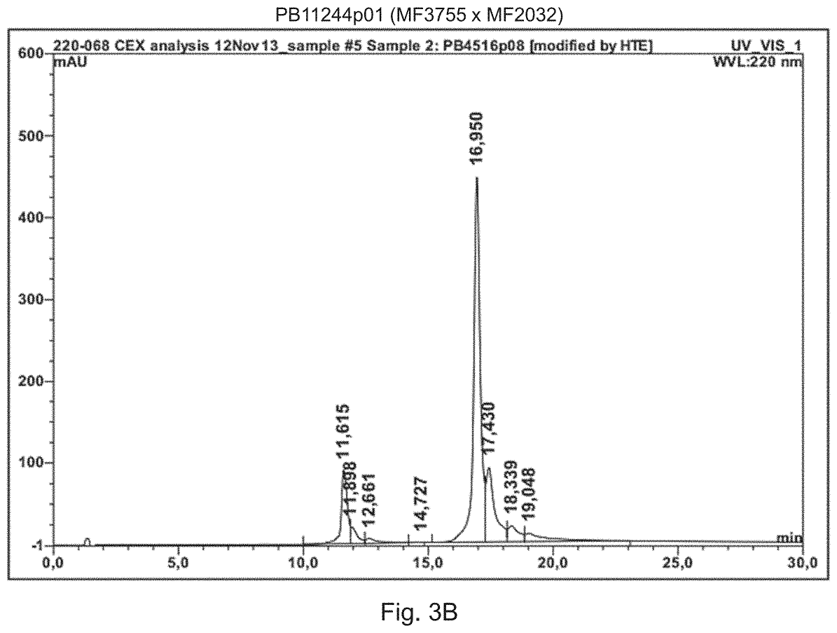

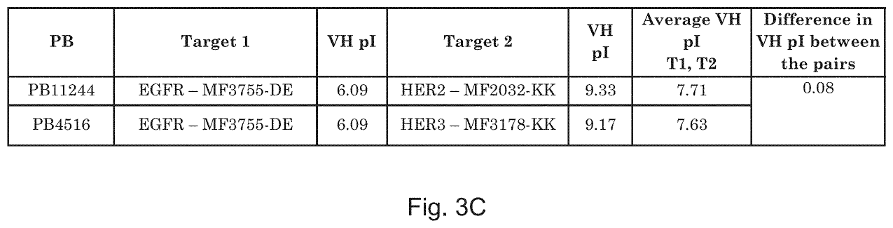

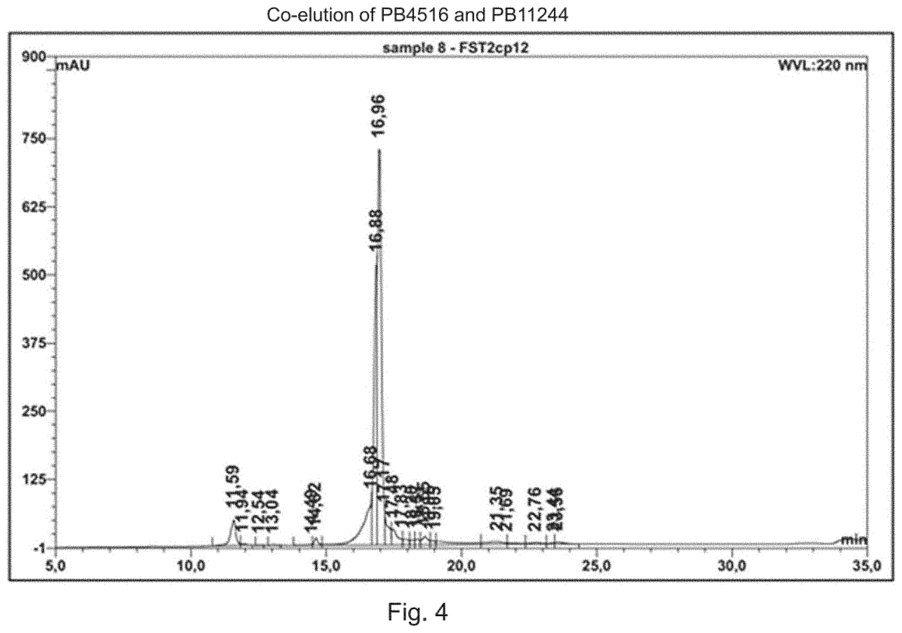

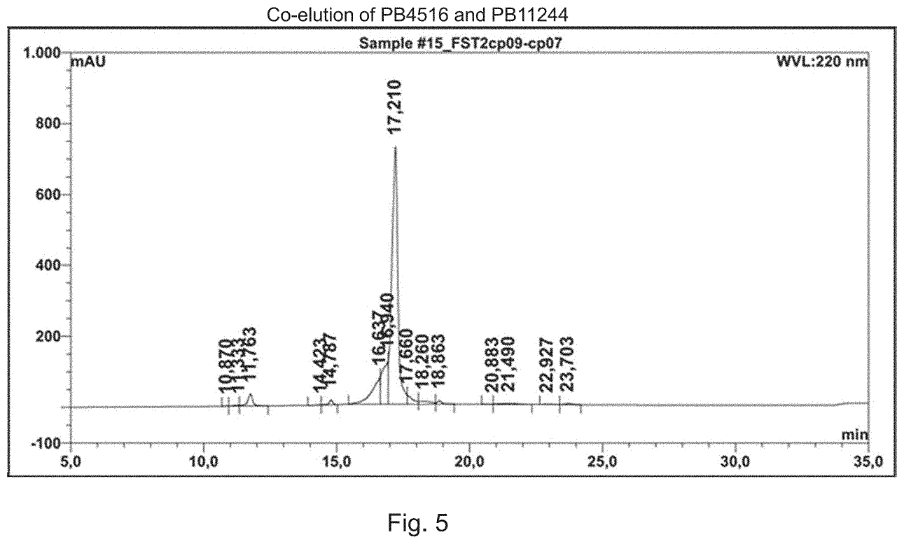

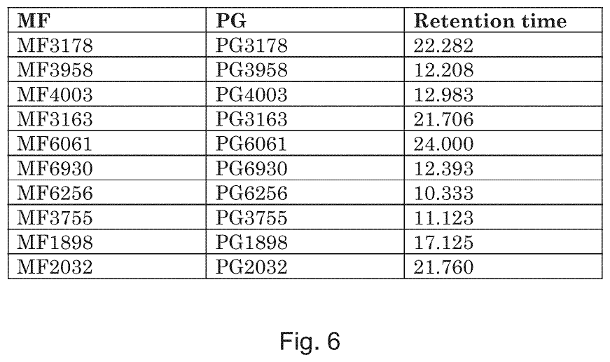

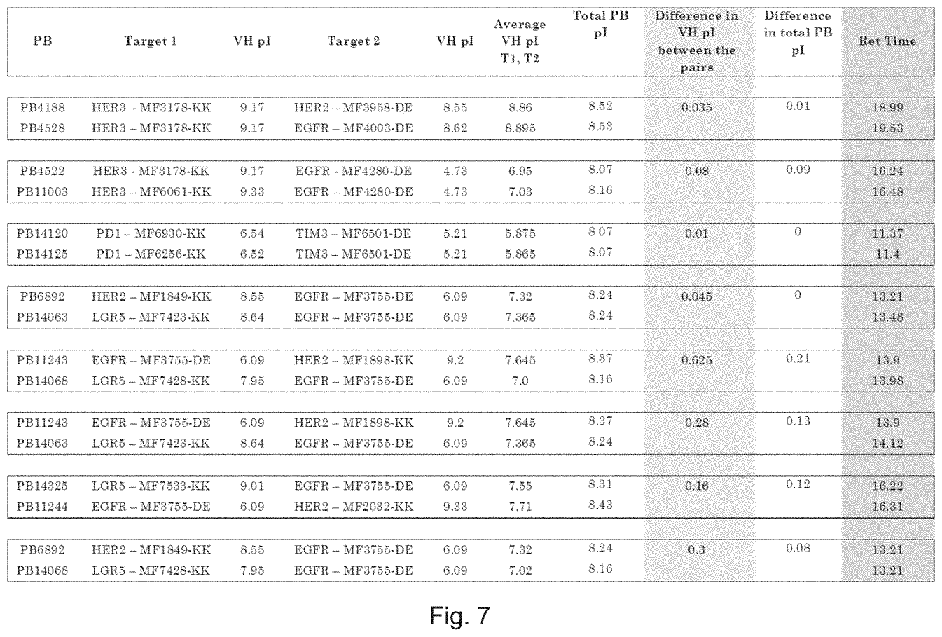

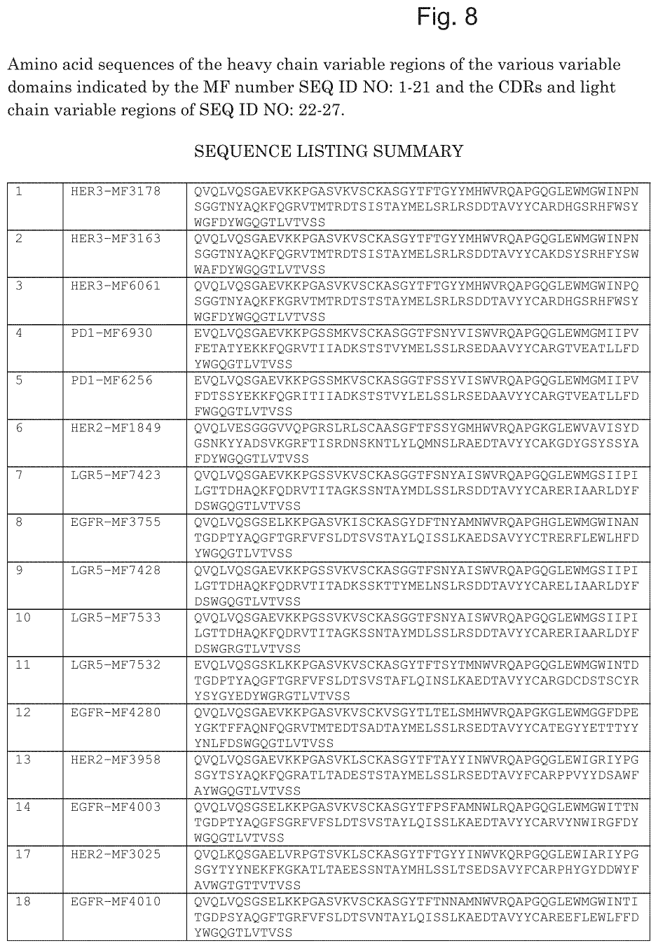

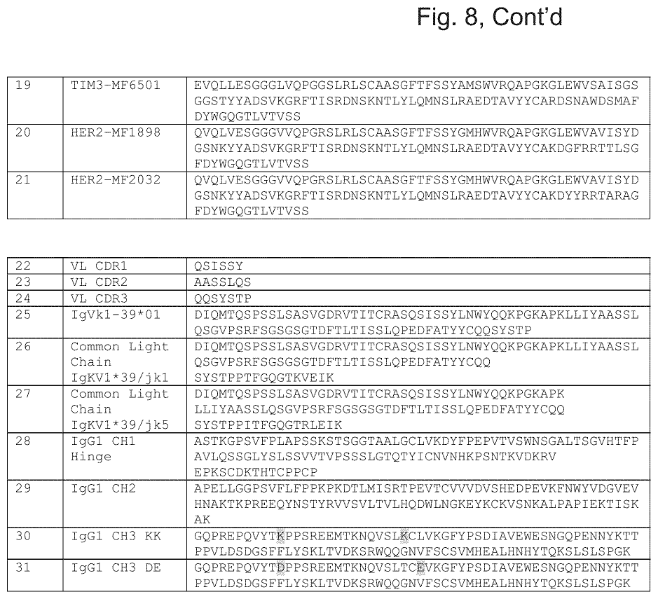

A schematic representation of embodiments wherein the composition comprises two bispecific antibodies that share a common arm. The figure depicts antibodies with heavy chains (1) and light chains (4). The four heavy chains have three different variable regions (5, 6 and 7). The heavy chain that has the shared variable region (5) has one part (3) of a heterodimerization domain. The heavy chains with variable regions (6) and (7) have the compatible part of the heterodimerization domain (2). Preferred pairing of heterodimerization regions (2) and (3) can direct formation of bispecific antibodies.

FIG. 2

Panel A: CIEX-profile at 220 nm of bispecific antibody PB4516 production number 8 (p08). Panel B: CIEX-profile at 220 nm of bispecific antibody PB6892 production number 4 (p04).

FIG. 3

FIG. 3a: CIEX-profile at 220 nm of bispecific antibody PB4516 production number 10 (p10).

FIG. 3b: CIEX-profile at 220 nm of bispecific antibody PB11244 production number 1 (p01).

FIG. 3c: Per box two bispecific antibodies are identified in the column PB (PBXXXX(X)). The heavy chain variable region for PB11244 and PB4516 is indicated in the columns Target 1 and Target 2. The sequence of the light chain is the same for all antibodies and has the amino acid sequence of the common light chain IgKV1*39/jk1 of SEQ ID NO: 26. The pI calculated with the ExPASy. ProtParam tool is indicated for each heavy chain variable region in the columns pI. The pI difference between the two heavy chain variable regions is indicated in the last column, demonstrating that the average VI pI differential between PB11244 and PB4516 is 0.08.

FIG. 4

CIEX-profile at 220 nm of an antibody preparation of individual colony cp12 of pool FST2. The CIEX-profile shows a sharp peak of co-eluting antibodies PB4516 and PB11244. The profile shows that the sample contains a limited amount of product related impurities. It also shows the good separation between the co-eluting bispecific antibodies and the separately migrating product-related impurities.

FIG. 5

CIEX-profile at 220 nm of an antibody preparation of colony CP07. The colony was picked from a collection of individual colonies of single colony FST2cp09. The second subcloning was done to make sure that the FST2cp09-cp07 cell line was a clonal cell line. The bispecific antibody specific ELISA indicated the presence of 743 sg/ml of the ECFRIHER2 bispecific antibody PB11244 and 1134 μg/ml of the EGFR/HER3 bispecific antibody PB4516.

FIG. 6

Retention time of antibody homodimers (PGXXXX) having two identical variable domains. The amino acid sequence of the heavy chain variable region has the sequence indicated for the MF in FIG. 8 and a common light chain IgKV1*39/jk1 of SEQ ID NO: 26.

FIG. 7. Per box two bispecific antibodies are identified in the column PB (PBXXXX(X)). The heavy chain variable region (MFXXXX) of each of the bispecific antibodies is indicated in the columns Target 1 and Target 2. The sequence of the light chain is the same for all antibodies and has the amino acid sequence of the common light chain IgKV1*39/jk1 of SEQ ID NO: 26. The pI calculated with the ExPASy, ProtParam tool is indicated for each heavy chain variable region in the columns pI. The pI difference between the two heavy chain variable regions and the retention time measured are indicated in the last two columns. The measured retention times and the calculated pI and average pI indicate that the couple of bispecific antibodies can efficiently co-elute in CIEX chromatography. The antibodies have IgG1 constant regions and a common light chain. The heavy chain with the shared heavy chain variable region (identical MF) with the CH3 DE heavy chain or the KK heavy chain is indicated. Retentions times are indicated for CIEX chromatography performed on each bispecific, with an exemplary condition for CIEX chromatography described the materials and methods section. Many other CIEX chromatography conditions will result in suitable retention times between listed the bispecific antibody pairs having the provided pI values.

FIG. 8

Amino acid sequence of the heavy chain variable regions (MFXXXX) of respective antibodies and CDRs of light chain variable regions and amino acid sequence of common light chain variable regions.

### EXAMPLES

### Example 1

Materials and Methods

Cell Lines

HEK293 and CHO-K1 were maintained in growth medium.

Generation of Bispecific Antibodies

Bispecific antibodies were generated using the proprietary CH3 technology to ensure efficient hetero-dimerization and formation of a bispecific antibody. The CH3 technology uses charge-based point mutations in the CH3 region to allow efficient pairing of two different heavy chain molecules as previously described (PCT/NL2013/050294; published as WO 2013/157954 A1).

A VH gene was cloned in one of two different backbone IgG1 vectors. Depending on the binding partner the VH was cloned in an IgG1 backbone comprising the CH3 variant with heterodimerization variant. “DE” or in the IgG1 backbone comprising the complementary CH3 heterodimerization variant “KK”. In case of bi- or multispecific antibodies wherein two or more antibodies share a heavy chain. The shared chain preferably has the CH3 heterodimerization variant “DE” (also referred to as the DE-heavy chain) and the two or more unique heavy chains have the CH3 heterodimerization variant “KK” (also referred to as the KK-heavy chains).

HEK293 cells were transiently transfected with the DNA-FUGENE mixtures and further cultivated. Seven days after transfection, supernatant was harvested and medium was refreshed. Fourteen days after transfection supernatants were combined and filtrated through 0.22 μM. The sterile supernatant was stored at 4° C. Suspension adapted 293F cells were cultivated in T125 flasks at a shaker plateau until a density of 3.0×10e6 cells/ml. Cells were seeded at a density of 0.3-0.5×10e6 viable cells/ml in each well of a 24-deep well plate. The cells were transiently transfected with the individual sterile DNA: PEl-MIX and further cultivated. Seven days after transfection, supernatant was harvested and filtrated through 0.22 μM. The sterile supernatant was stored at 4° C.

Generation of Stable Cell Line Pools that Co-Express Two Bispecific Antibodies

CHO cells were transfected with the three heavy chain constructs and a common light chain construct in a molar ratio of common light chain construct (cLC):EGFR heavy chain:HER2 heavy chain:HER3 heavy chain=2.5:2:1:1. Ten pools of stably transfected cells were obtained (A-J). ELISA analysis of anti-EGFR, anti-HER2 and anti-HER3 antibodies was performed on the day 3 and day 6 supernatants of the 10 pools. All 3 species could be detected in all pools.

Generation of Stable Cell Line Clones that Co-Express Two Bispecific Antibodies.

The pools were plated in semi-solid medium and allowed to grow for 7-10 days. Single colonies were picked and seeded into 24 well culture plates. Colonies were reseeded prior to collection of antibodies from the supernatant of the cultures.

Determination of Antibody Titers

Anti-HER2 antibody titers of samples containing a single bispecific antibody were determined by ELISA against Erbb-2 Fe protein (R&D systems). Anti-HER3 titers of samples containing a single bispecific antibody were determined by ELISA against human Erbb-3-Fe protein (R&D systems). Anti-EGFR antibody titers of samples containing a single bispecific antibody were determined by ELISA against human EGFR ECD-Fc protein (R&D systems). Serial 2-fold dilutions of the antigens were used to coat wells of an ELISA plate, starting at 5 μg/ml.

ELISA assays to quantify EFGR×HER2 and EGFR×HER3 bispecific antibodies in compositions comprising mixtures of the two bispecific antibodies were done by coating ELISA plates with EGFR-Fc (R&D systems). After washing plates were incubated with sample. After washing the presence of bound bispecific antibody with one EFGR arm and one HER2 arm was detected by incubating with labelled HER2-Fc. The presence of bound bispecific antibody with one EFGR arm and one HER3 arm was detected by incubating with labelled HER3-Fc.

IgG Purification

Purification of IgG was performed using affinity chromatography. Purifications were performed under sterile conditions using vacuum filtration. First the pH of the medium was adjusted to pH 8.0 and subsequently the productions were incubated with protein A Sepharose CL-4B beads (50% v/v) (Pierce) for 2 H at 25° C. on a shaking platform at 600 rpm. Next the beads were harvested by vacuum filtration. Beads were washed twice with PBS pH 7.4. IgG was eluted at pH 3.0 with 0.1 M citrate buffer and the IgG fraction was immediately neutralized by Tris pH 8.0. Buffer exchange was performed by centrifugation using Ultracel (Millipore). The samples ended up in a final buffer of PBS pH 7.4.

Cation-Exchange Chromatography (CIEX)

CIEX-HPLC chromatography was done using TSKgel SP-STAT (7 μm particle size, 4.6 mM I.D.×10 cm L, Tosoh 21964) series of ion exchange columns. The columns are packed with non-porous resin particles for speed and high resolution analysis, as well as isolation, of biomolecules. The particles in TSKgel STAT columns contain an open access network of multi-layered ion-exchange groups for loading capacity, while the particle size makes these columns suitable for HPLC and FPLC systems.

The TSKgel SP-STAT (7 μm particle size, 4.6 mM I.D×10 cm L, Tosoh 21964) is equilibrated using Buffer A (Sodium Phosphate buffer, 25 mM, pH 6.0), after which antibodies are displaced from the column by increasing salt concentration and running a gradient of Buffer B (25 mM Sodium Phosphate, 1 mM NaCl, pH 6.0). Flow rate was set at 0.5 mL/min. The injection sample mass for all test samples and controls (in PBS) was 10 μg and injection volumes 10-100 μl. The chromatograms are analyzed for peak patterns, retention times and peak areas for the major peaks observed based on the 220 nm results.

Results

The CIEX profiles of the bispecific antibodies PB4516p08 and PB6892p04 were compared (see FIG. 2). It was observed that the production of PB6892 contained a significant amount of impurities. Also the retention time of the bispecific antibody fraction of PB6892 was significantly lower than the retention time of the bispecific antibody fraction of PB4516. For co-production by the same cell and subsequent co-purification using CIEX the retention time the two bispecific antibodies are preferably closer together. For this reason the variable region of the HER2 arm for PB6892 was replaced by a different variable region. The heavy chain with the variable region MF2032 was selected and used to produce the EGFR×HER2 bispecific antibody PB11244. The CIEX profiles of P14516p10 and PB11244p01 are shown in FIG. 3. The retention times of the bispecific antibody fractions are respectively 16.310 and 16.950. These retention times are sufficiently the same to allow for co-purification using CIEX under the conditions indicated. In addition, the figure shows that the retention times of impurities are sufficiently different to allow efficient separation in analytical and preparative columns. The above bispecific antibody preparations were produced in HEK293 cells.

For co-production CHO-K1 cells were used. CHO cells were transfected with constructs containing the three heavy chains with the respective variable regions of MF3755 (EGFR), M20:32 (HER2) and MF3178 (HER3) were transfected into CHO-K1 cells together with a construct expression the light chain variable region of SEQ ID: NO: 26. Vector positive cells were selected and pooled. Ten separate pools of transfected CHO-K1 cells (identified A-J) were generated.

Table 1 shows the amounts of bispecific antibody PB34516 (EGFR×HER3) and PB11244 (EGFR×HER2) produced by the respective pools. Also the ratio of the amounts as well as the total amount of IgG produced is shown. Pools F and J were selected for subcloning.

Table 2 shows the amount of bispecific antibody PB34516 (EGFR×HER3) and PB11244 (EGFR×HER2) produced by the respective clones.

Antibody produced by clone FST2cp12 was used to analyze the CIEX profile (see FIG. 4). It is clear that the two bispecific antibodies efficiently co-elute in the same CIEX elution fractions.

Clone FST2cp09 was further subcloned to make sure that the cell line was clonal and a further CIEX profile was determined of the antibodies produced. FIG. 5 shows the CIEX profile. It is clear that the two bispecific antibodies efficiently co-elute in the same CIEX elution fractions. The relative contribution of the two bispecific antibodies in the co-elution is analysed by ELISA and/or by hydrophobic interaction columns. The bispecific antibody specific ELISA indicated the presence of 743 μg/ml of the EGFR/HER2 bispecific antibody PB11244 and 1134 μg/ml of the EGFR/HER3 bispecific antibody PB4516.

### Example 2

Generation of Stable Cell Line Pools that Co-Express Two Bispecific Antibodies

The cell lines expressing the two by two bispecific antibodies listed in FIG. 7 are produced as follows. CI cells are transfected with three heavy chain constructs and a common light chain construct. The three heavy chains are identified by the heavy chain variable regions (MFXXXX) indicated in the box. The light chain comprises the light chain variable region sequence of IgVk1*39/jk1 of SEQ ID NO: 26. The two bispecific antibodies have one heavy chain in common and one different heavy chain each. For instance, the first couple specified in FIG. 7 share a common heavy chain comprising the same HER3 binding arm comprising a heavy chain variable region (MF3178) and a different second binding arm. PB4528 has an EGFR binding arm with a heavy chain variable region (MF4003) and PB4188 has a HER2 binding arm with a heavy chain variable region (MF3958). The shared heavy chain has the KK CH3 region of the compatible DE/KK heterodimerisation domain. The shared heavy chain arm may also have the DE C13 region. For example, at FIGS. 3-5, two bispecific antibodies are co-purified PB11244 and PB4516. As shown at FIG. 3c, PB11244 and PB4516 share the same EGFR binding arm with a heavy chain variable region (MF3755) and PB11244 has a HER2 binding arm with a heavy chain variable region (MF2032) and PB34516 has a HER3 binding arm with a heavy chain variable region (MF3178). The shared heavy chain arm in this pair of bispecific antibodies have the DE CH3 region, while the different HER2 and HER3 binding arms have the KK C13 region.

The molar ratio of common light chain construct (cLC):to shared heavy chain construct:to different heavy chain construct 1:different heavy chain construct 2=2.5:2:1:1. Pools of stably transfected cells are obtained. ELISA analysis of the antigens is performed on supernatants collected from the pools. All 3 antigen binding species are detected in the pools. The CIEX retention times of the bispecific antibodies in each couple of FIG. 7 are determined under similar CIEX conditions and indicated in the 8^{th} column. The deviation from the average retention time is calculated with the formula 100×((A−B)/(A+B)), where A is the retention time of the bispecific antibody with the longest retention time. For example, the deviation for the first couple is 100×((16.46−16.24)/(16.46+16.24))=0.67 or 0.7%.

Antibodies in the collected supernatants are first separated from other proteins in the supernatant by protein A extraction followed by acid elution and quick neutralization. The buffer of the collected antibodies is subsequently exchanged for PBS. The samples are subsequently loaded onto CIEX columns and washed and eluted by imparting an increasing salt gradient. The absorption of the eluate is measured at 220 nm and the retention times are calculated from the start of the salt gradient and the observance of the peak(s) for the bispecific antibodies. The bispecific antibodies are collected and the respective bispecific antibodies in the collected eluate is verified by ELISA. The retention times for the respective bispecific antibodies are indicated in the last column. It is clear that many of the couples have retention times that co-elute efficiently in a CIEX column. It is also clear that the CIEX chromatography provides a good separation of the co-eluting bispecific antibodies and the respective homodimers (if any). FIG. 6 lists the retention times of various antibodies with homodimers of heavy chains comprising the heavy chain variable regions present in co-eluting bispecific antibodies. It is clear that the retention time of the homodimers is sufficiently different from the retention times of the respective bispecific antibodies. For instance in the first box of FIG. 7 the homodimers PG3178. PG3958 and PG4003 could be present in a production. Retention times of the respective homodimers are about 22, 12 and 13 (FIG. 6 rows 1-3), whereas the retention times of the bispecific antibodies comprising the heavy chain variable regions are about 19 and 19.5 (FIG. 7, rows 1 and 2).

TABLE 1
Quantification of EGFR × HER2 and EGFR × HER3 bispecific
antibody production in pools of cells. The culture supernatants
of ten pools (A-J) were evaluated. The ELISA assays were based
on EGFR-Fc coating, binding of antibody produced and detection
with either labelled HER2-Fc or labelled HER3-Fc. Pools A-J were
analyzed using the two ELISA assays. The bispecific antibodies
PB4516 and PB11244 are IgG1 heavy chain antibodies with
compatible DE/KK heterodimerization domains. The heavy chains
are combined with the common light chain. The bispecific antibodies
share the MF3755 heavy chain variable region on one heavy chain
and each have a different heavy chain variable region on the other
IgG1 heavy chain, MF3178 for PB4516 and MF2032 for PB11244.

| | [symbol] | [symbol] | Ratio | Total |
|---|---|---|---|---|
| | [symbol] | [symbol] | [symbol] | [symbol] |
| Pool | [symbol] | [symbol] | [symbol] | [symbol] |
| A | 153.6 | 162.0 | 1:1.1 | 315.6 |
| B | 120.1 | 336.3 | 1:2.8 | 456.3 |
| C | 325.5 | 741.6 | 1:2.3 | 1067.1 |
| D | 510.6 | 856.8 | 1:1.7 | 1367.4 |
| E | 273.0 | 526.5 | 1:1.9 | 799.6 |
| F | 1824.7 | 1024.9 | 1:0.6 | 2849.6 |
| G | 704.2 | 849.8 | 1:1.2 | 1554.0 |
| H | 951.1 | 467.5 | 1:0.5 | 1418.6 |
| I | 450.7 | 691.0 | 1:1.5 | 1141.7 |
| J | 1288.9 | 1572.1 | 1:1.2 | 2861.0 |

[symbol]

indicates data missing or illegible when filed

TABLE 2
Selected pools were used for single cell cloning. 18 colonies were
picked from three pools. Two independent pools F (FST1 and FST2)
and one pool J (JST1) were used for single cell cloning. The indication
“cp” followed by a number identifies individual colonies of a pool.
Picked colonies were grown up and used for the collection of
antibodies. Individual colonies from the same pools produced different
amounts and different proportions of the respective bispecific antibodies.

| # | HER2 [ug/mL] | HER3 [ug/mL] | HER2/HER3 |
|---|---|---|---|
| FST1cp02 | 48.7 | 1449.2 | 0.03 |
| FST1cp03 | 103.4 | 488.9 | 0.21 |
| FST1cp04 | 131.4 | 1675.0 | 0.08 |
| FST1cp14 | 259.1 | 214.6 | 1.21 |
| FST1cp24 | 372.3 | 817.0 | 0.46 |
| FST1cp26 | 92.2 | 706.7 | 0.13 |
| FST2cp09 | 1026.2 | 1509.0 | 0.68 |
| FST2cp12 | 725.7 | 1334.2 | 0.54 |
| FST2cp13 | 737.4 | 1173.0 | 0.63 |
| FST2cp20 | 617.6 | 1759.3 | 0.35 |
| FST2cp21 | 993.0 | 1852.3 | 0.54 |
| FST2cp23 | 937.4 | 1095.5 | 0.86 |
| JST1cp01 | 121.3 | 490.6 | 0.25 |
| JST1cp04 | 239.8 | 383.7 | 0.62 |
| JST1cp05 | 187.2 | 759.0 | 0.25 |
| JST1cp09 | 828.6 | 718.5 | 1.15 |
| JST1cp13 | 103.5 | 175.8 | 0.59 |
| JST1cp24 | 481.5 | 423.7 | 1.14 |

1. A method of producing at least two antibodies comprising
providing cells with nucleic acid that encodes the antibodies;
culturing said cells;
collecting the antibodies from the culture; and
separating produced antibodies from half antibodies by ion exchange chromatography (IEX);
the method characterized in that the antibodies exhibit IEX retention times that that deviate by 10% or less from the average of the retention times of the individual antibodies under the IEX conditions used.

2. The method of claim 1, wherein the collection of the antibodies from the culture comprises purifying antibody from other proteins by antibody affinity purification, preferably by protein A extraction.

3. The method of claim 2, wherein further comprising subjecting affinity purified antibodies to size-exclusion chromatography (gel-filtration chromatography and/or anion-exchange chromatography.

4. The method of claims 1-3, wherein subsequent to the IEX the collected antibodies are quantitatively analyzed for relative expression levels by hydrophobic interaction chromatography (HIC).

5. The method of claim 4, wherein the specificity of the collected antibodies is verified by ELISA.

6. The method of claims 1-5, wherein the retention times of the respective half antibodies are outside the range spanned by the retention times of the antibodies.

7. The method of claim 6, wherein the cells produce 3 heavy chains.

8. The method of claim 7, wherein said heavy chains comprise domains for efficient heterodimerization of heavy chains.

9. The method of claim 1-8, wherein at least two of said antibodies are bispecific antibodies.

10. The method of claim 1-9, wherein at least two of said antibodies share an identical heavy chain.

11. The method of claims 1-10, wherein said antibodies have isoelectric points (pI) that differ by 0.4 units or less from the average pI of said at least two antibodies.

12. The method of claims 1-11, wherein the antibodies are selected for having heavy and light chain combinations that have retention times that are significantly different from the retention times of the full antibodies under the IEX conditions used.

13. The method of claim 12, wherein the pI of heavy and light chain combinations differ by more than 0.4 units from the average pI of said at least two antibodies.

14. The method of claims 1-13, wherein the heavy chains comprise a CH3 domain that favors heterodimerization of heavy chains.

15. The method of claim 1-14, wherein the heavy chains of said antibodies are IgG heavy chains.

16. The method of claims 7-15, wherein one heavy chain comprises the amino acid substitutions L351K and T366K (EU numbering) in the CH3 region and another heavy chain comprises the amino acid substitutions L351D and L368E in the CH3 region.

17. A method of producing at least two antibodies comprising
providing cells with nucleic acid that encodes the antibodies;
culturing said cells;
collecting the antibodies from the culture; and
separating produced antibodies from half antibodies by ion exchange chromatography (IEX):
the method characterized in that the antibodies exhibit IEX retention times that that deviate by 10% or less from the average of the retention times of the individual antibodies under the IEX conditions used and wherein subsequent to the IEX the collected antibodies are quantitatively analyzed for relative expression levels by hydrophobic interaction chromatography (HIC) and the specificity of the collected antibodies is verified by ELISA.

18. A composition comprising 2-10 recombinant antibodies obtainable by a method of claims 1-17.

19. A composition comprising 2-10 recombinant antibodies characterized in that the IEX retention times of at least two of said antibodies deviate by 10% or less from the average of the retention times of the individual antibodies under the IEX conditions.

20. A composition comprising 2-10 recombinant antibodies characterized in that the pI of at least two of said antibodies differ by 0.4 units or less from the average pI of said at least two antibodies.

21. The composition of claims 18-20, characterized in that the IEX retention times and/or the pI are essentially the same for all of the antibodies.

22. The composition of claims 18-21, wherein at least two of the antibodies are bispecific antibodies.

23. The composition of claim 22, wherein at least two of said antibodies share an identical heavy chain.
